(19) United States
(12) Patent Application Publication
(10) Pub. No.: US 2020/0188181 A1
(43) Pub. Date: Jun. 18, 2020

(54) Adhesive Bandage with Disposable Abrasive Facings Assembly and a Method of Using the Same

(71) Applicants: Cristy Elliott, Canton, TX (US); Joel Elliott, Canton, TX (US)

(72) Inventors: Cristy Elliott, Canton, TX (US); Joel Elliott, Canton, TX (US)

(21) Appl. No.: 16/219,109

(22) Filed: Dec. 13, 2018

(51) Int. Cl. A61F 13/02; A61L 15/58; A45D 29/04

(52) U.S. Cl. CPC A61F 13/0206; A61F 13/0246; A61F 2013/00531; A45D 29/04; A61L 15/58

(57) ABSTRACT

The current invention enhances the disposable face or facings on any commonly used adhesive bandage into a usable nail file and callus remover along with preserving its original function of protecting the adhesive layer on the bandage. The current invention eliminates the need to use a separate manicure tool, nail file, or callus remover when the shaping and exfoliating tools are contained on the disposable face or facings of the adhesive bandage. This efficiently allows the user to maximize the utilization and function of not only the adhesive bandage but also the disposable face or facings. Because the disposable facings may be altered at minimal cost to include the abrasive surface, the apparatus is a significant improvement over the current products on the market.

### BACKGROUND OF THE INVENTION

This invention relates to abrasive materials and methods used for filing human nails, callouses, or any variety of skin irritations and particularly, an adhesive medicated bandage with disposable abrasive face or facings which comprise an abrasive surface used to file calloused feet, elbows, hands, human nails or any callused skin.

It is commonly known that nail scissors, fingernail files, pumice stones, and other smoothing tools used in manicure treatments are readily available for professional nail care specialists and the general public. These manicure tools each have a specific mechanism for shaping and reshaping fingernails and callouses. The degree of care needed to shape the surface of the fingernail generally determines which manicure treatment and tool to employ. Nail files are generally used to round, shorten, or define nails and calluses on both the feet and hands of homosapiens.

A nail file is generally a manicure tool that only functions as a nail file. Similarly, a pumice stone is a generally a manicure tool that only functions as a smoothing treatment tool. In other words, a nail file and a pumice stone are both stand-alone pieces of manicure equipment with one singular function. The nail file function is for filing human nails or callouses. The pumice stone function is to smooth rough patches of skin. If any additional treatments to the nail or affected skin is required to address and repair the skin issue, an additional tool is necessary to repair the infected area.

Common nail files are made up of an abrasive layer of sand paper like coating mounted on opposite sides of a rigid and elongated structure. The abrasive surface may contain diamond particulate to intensify the filing effectiveness. The entire structure and parts of the nail file are made of inexpensive material when the nail file is intended to be a limited use disposable nail file.

Other nail files are also available that are more expensive due to the upgraded durability of the rigid and elongated structure that extend the life of the file. Professional nail care specialists frequently utilize the more expensive nail files while the general public generally utilizes the disposable inexpensive nail files. Both nail file types come in all shapes and sizes based on the customer's preference.

Common nail files' functionality is limited due to their rigid shape. The rigid shape limits the flexibility of the file, which in turn limits the surfaces the file can effectively operate. The rigid shape also may not necessarily allow the file to be positioned to insure the most effective operation or functioning thereof. The rigid structure of the common nail file does not allow the user to reach every surface that requires treatment. Although rigid nail files are mostly effective in filing fingernails and easy to reach affected skin, it is desirable to increase the flexibility of the filing surface which will broaden the affected skin areas the file can reach and smooth.

In addition to the limited areas a common nail file can reach effectively, germs are also an additional issue the user should be aware of in order to prevent infections spreading to the affected skin. Fungus and other types of infections can impregnate nail files when used repeatedly over a period of time. Both expensive and inexpensive nail files could be infectious carriers after continuous use.

Professional manicurists often soak their more durable and expensive files in a sanitizing agent to prevent the spread of infection but the general public does not typically sanitize their nail files. Additionally, nail files commonly found in the market place do not contain a mechanism or means themselves to prevent or detect the presence of an infection. Both expensive and inexpensive nail files when worn or soiled should be thrown away in order to minimize the risk of infection because the worn or soiled files could be impregnated with infections regardless of how much use is left in the file itself. Once soiled, the file should be disposed of appropriately. Holding onto the file until the abrasive particles do not function properly, potentially increases the risk of spreading infection because the file may be a carrier for toxicities. Decreasing the spread of infection and limiting the prolonged use of the files are issues that the present invention remedies.

Nail files, exfoliating files, abrasive files, pumice stones and other abrasive materials and methods are commonly used on calluses, corns, rough patches of skin, hangnails, blisters, and dry skin in addition to human nails to smooth the affected area. The user applies the desired amount of pressure on the file itself or pumice stone itself directly to the affected area of skin to smooth and eliminate any unwanted rough patch or skin issue that produces an uncomfortable area on the body by moving the filing apparatus back and forth over the affected area or areas. The abrasive coating allows the user to gently file down and/or exfoliate the affected area to expose a smoother healthier layer of skin. The user is able to select the degree of filing by selecting the level of abrasion on the file, by the amount of pressure the user applies to the file, and the duration of filing the affected area. The degree of irritation and skin issue to be addressed also factors into the abrasion selection.

Issues on the skin develop on both male and female skin for a variety of reasons. Friction, rubbing, and pressure can cause blisters, calluses and corns on hands, feet, or any area of skin exposed to these elements. Left untreated, these skin issues often progress and worsen without proper treatment. Females are more likely than males to have a nail file or manicure tool in their purse to address the issue. The average male does not customarily carry nail files with them on a daily, monthly or yearly basis. A simple inexpensive file when readily available can treat the skin irritation quickly and often painlessly but left untreated, can cause infection and pain. Equipping men and women to address these skin issues quickly, will help prevent further injury and decrease the risk of the skin issue infected.

The first aid kit on a job site more often than not will not contain a nail file. As a result, males are less likely to immediately treat the affected area but instead wait until the affected area is infected to seek treatment. Most first aid kits contain several cloth bandages and adhesive bandages in a variety of shapes, sizes, and treatment capacities. A bandage helps protect the affected skin but does not have any mechanism itself to address the abrasion or skin issue on the affected area. Additional tools are needed to treat the affected area are typically not be contained in a first aid kit.

Items commonly found in first aid kits expectedly contain bandages, sponges, gauze, and ointment to treat minor skin issues. Additionally, scissors, tweezers, creams, bandages, and gauze dressings are some of the items commonly found in a first aid kit. However, one item that is commonly missing that proves to be very useful in treating skin issues is a manicure tool that contains an abrasive surface that can treat the affected skin needing repair. Many times the affected skin would benefit from the filing the affected area before placing a bandage over the infected area. The addition of this a tool that comprises this filing ability to smooth the affected skin to a first aid kit would be beneficial for treating skin issues that can be addressed and completed by the injured themselves.

Bandages come in all shapes, sizes, thickness, and application. Bandages can be general-purpose usage or designed for very specific application and use. Regardless of the designed use, self-adherent bandages contain disposable faces that stick to the adhesive layer of the bandage to protect the adhesion until time of desired use. The adhesion layer is essential for the bandage to stick to the skin but the faces that protect the adhesive are a single function, single use disposable inexpensive material. The faces themselves serve only to protect the adhesive layer and no function outside of the adhesion protection. Because every self-adhesive bandage contains disposable faces, it would be desirable to keep manufacturing costs low while giving the faces an additional function that would benefit the bandage user.

Bandages additionally can be pretreated with a variety of medicine, lotion, or ointments to treat almost any issue on the skin. Antibacterial formulations, skin smoothing lotions, blister specific medicine, and a variety of other medicated bandages are commonly found on the market. These products combine two essential treatments needed to protect and treat affected skin irritations or cuts. The current invention greatly improves the market and treatments available for affected skin by combining an abrasive material to the medicated bandage for additional treatment options to smooth and improve the skin prior to applying the medicated bandage to the skin. The additional treatment method improves the overall treatment regime currently available on the market.

### SUMMARY OF THE INVENTION

It is the objective of this invention to provide an abrasive material capable of filing assembly enclosed on disposable face or faces, which are contained on a self-adhesive bandage that provide a multifunction filing apparatus for the purpose of treating a fingernail or affected skin, such as rough smoothing calloused, flaking, peeling, scaly or dry skin or any other nail or skin issue that requires exfoliation to final gloss polishing.

This invention has several features; no single one of them is solely responsible for its desirable attributes. By no means limiting the scope of the invention as expressed by the information which follows, the inventions more prominent features will now be discussed briefly. After reviewing the summary and detailed description, one will understand how the features of this invention provide its benefits, which include providing the disposable faces with a separate and distinct function from merely protecting the adhesive layer on a bandage, providing an abrasive substance capable of filing in conjunction with a bandage in order to increase the quality of care provided by a self-adhesive bandage's overall function, and increase availability of an abrasive substance capable of filing by combining the abrasive surface capable of filing within a self-adhesive bandage.

The first feature of this invention is that it includes a disposable face having an abrasive surface. The face may be made of paper, plastic, fabric, or plastic foam. The face is covered with a film on one side that provides the abrasive surface. The opposite side of the face is smooth and does not contain any abrasive material. The shape of the face can be round, square, rectangle, or any shape necessary to cover the adhesive section on the self-sticking bandage.

Another feature of this invention is to provide an abrasive surface filing apparatus which comprises one face or a plurality of disposable filing faces each including a coating of abrasive media of varying grain sizes applied to one side of the face or one side of a plurality of faces.

Another feature of the present invention is to provide an abrasive surface filing apparatus, which comprises one or a plurality of abrasive disposable faces, each carrying a coating of filing material composed of a mixture of hard fine particle substances of varying grain size.

A further object of the present invention is to provide an abrasive surface filing apparatus, which includes different levels of abrasion on the coating of filing material on one face or a plurality of disposable faces needed according to the degree of smoothing treatment needed on the skin or nail.

A further object of the present invention is to provide an exfoliating filing apparatus, which includes different levels of abrasion on the coating of filing material on one face or a plurality of disposable faces needed according to the degree of smoothing treatment needed on the skin or nail.

A still further object of the present invention is to provide a disposable abrasive surface filing apparatus with minimal cost along with increased availability.

Traditional adhesive bandages frequently used in the market place contain disposable removable faces that protect the adhesion on the bandage apparatus when the faces are attached and secured to the apparatus. In the current marketplace, these disposable faces only serve one function that is to protect the adhesion on the bandage apparatus so that the bandage's adhesive remains secured and protected until time of use. The current invention improves the disposable face's function by transforming the single function face into a multifunction usable face.

The current invention renovates the disposable face into a usable disposable abrasive surface capable of filing and removing calluses while preserving its original function of protecting the adhesive layer on the bandage. The current invention eliminates the need to use a separate manicure tool, nail file, or callus remover because these shaping tools filing functions are contained on the disposable faces of the adhesive bandage as a combination apparatus. This apparatus efficiently allows the user to maximize the utilization and function of not only the adhesive bandage but also the disposable faces. Because the disposable faces may be altered at minimal cost to include the abrasive surface, the apparatus is a significant improvement over the current products on the market.

The current invention improves the overall filing ability for the user from the rigidity of a traditional nail file that is limited in the areas of skin that can be reached by the traditional nail file. The current invention is a flexible, pliable, shapeable apparatus that contains the abrasive surface on a removable face or faces. The entire structure of the present invention allows the user to bend, shape, fold, or manipulate the abrasive surface to reach areas the rigid traditional nail file cannot reach. The entire abrasive surface of the present invention can be rounded or manipulated in a variety of ways to the exact shape of the affected skin abrasion to treat the skin issues. After the affected skin is filed or smoothed to the user's desired level, the disposable faces or facings may be removed and the adhesive bandage applied to the protect the affected skin from further injury.

The present invention is made up of the combination apparatus comprising the exfoliating file and adhesive bandage that merges two separate and distinct tools into one combination economical disposable sanitary product. The present invention is designed to be a one-use application which dramatically reduces the spread of infection when compared to traditional nail file and/or exfoliating file.

When a traditional nail file is used repeatedly, bacteria and infection often impregnates the file. Using the traditional nail file on a variety of surfaces on a repeated basis often spreads infections unknown to the user. The present invention eliminates the spread of infection when used once on the affected area and disposed of after use. Because of the increased availability of the nail file being located on the disposable face of the bandage, the user is more likely to only use the abrasive surface on the face of the bandage once, discard it, and use a new disposable face on the next usage needed.

The present invention of the combined apparatus will be more readily available to the general public because the abrasive surface is contained on commonly found adhesive bandages, of which are found in all useful first aid kits. The common nail file is not frequently found in first aid kits nor is it a recommended item to keep in a first aid kit. The present invention solves this issue of not having a filing apparatus available in the commonplace first aid kit by adding the filing function and its benefits of filing to adhesive bandages found in almost every first aid kit.

The present invention may be customized to fit user's preferences. The adhesive bandage may or may not contain any variety of pretreated medication contained on the bandage. The medication contained on the bandage could be a general anti-bacterial treatment to prevent the affected area from becoming infected or the medication could be aromatherapy used solely to comfort the user's aromatic preferences.

For example, the present invention may be designed to file and smooth a construction worker's bloody, calloused, rough hands. After the calloused or damaged skin is repaired, the medicated bandage may be applied to prevent infection and protect the affected area so that the construction worker can return to work.

The present invention may also be designed for a homemaker who has a rough patch of skin on the heel of her foot that she wants to improve the appearance of by smoothing out the dead skin. After smoothing the troubled area, she may apply her bandage that contains lotions and aromatherapy to treat the affected area to improve the overall appearance. Either scenario may be accomplished with the present invention.

The present invention may also be designed for user to exfoliate an unwanted rough patch of skin. After the user smooths the skin to remove the troubled spot, the user removes the exfoliating materials from the filing apparatus exposing the pre-medicated section of the bandage. The user then places the pre-medicated bandage onto the skin for the amount of treatment time desired by user. User can select treatment time by wearing the bandage overnight or for a few hours.

The present invention greatly improves the current treatment options that are currently on the market. The combination exfoliating file and treatment located on the pre-medicated bandage provides the user a streamlined and simple treatment option all in one apparatus. Because the entire apparatus is disposable, the ease of use is also increased. User files and exfoliates rough patch of skin, places medicated bandage over rough patch of skin, and simply throws the entire apparatus away after desired use. The onetime use apparatus provides a sanitary option in one combination treatment apparatus. Moreover, the present invention is inexpensive and relatively easy to manufacture.

### BRIEF DESCRIPTION OF THE DRAWINGS

FIG. 1 is a top view of abrasive surface filing apparatus of the present invention in a stored state.

FIG. 2 is a bottom view of abrasive surface filing apparatus of the present invention in a stored state.

FIG. 3 is a perspective view of the abrasive surface filing apparatus of the present invention in a use position.

FIG. 4 is a side view of the abrasive surface filing apparatus of the present invention in a stored state.

FIG. 5 is a bottom view of abrasive surface filing apparatus of the present invention in a use position.

FIG. 6 is a side view of abrasive surface filing apparatus of the present invention in a use position.

### DETAILED DESCRIPTION

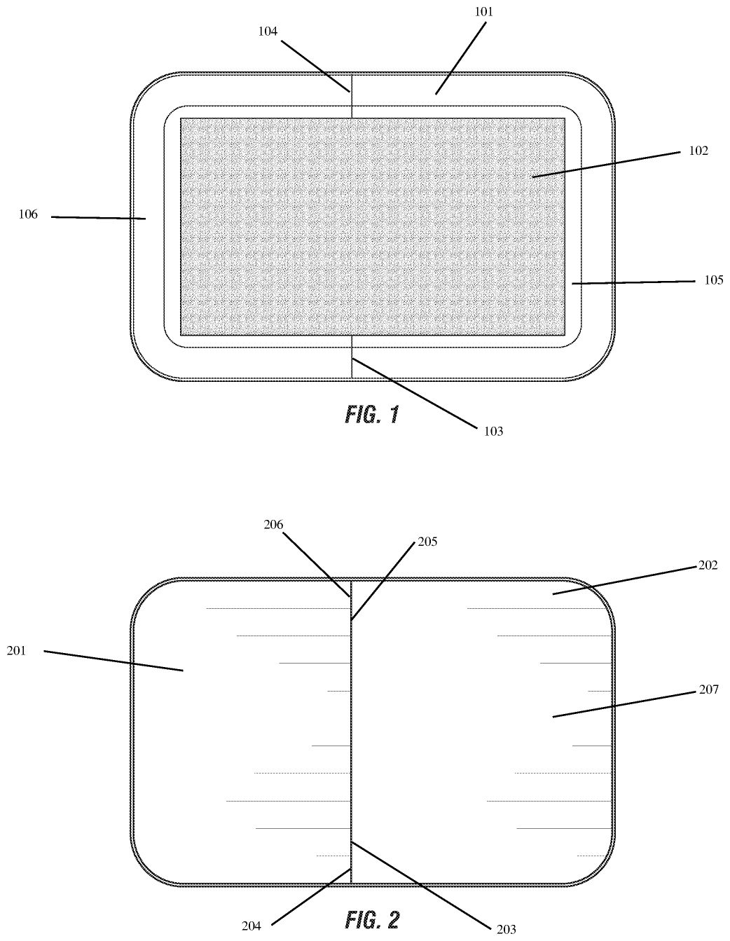

Applicants disclose, as illustrated in FIG. 1, the top view of abrasive surface filing apparatus of the present invention in a stored state. The top portion of the apparatus comprises a smooth elongated solid layer 101 consisting of an elastic type protective outer layer 101 that covers the entire top surface of the apparatus. The top portion 101 of the apparatus does not touch or contact the affected skin but instead protects the affected skin from the outer elements. The top portion of the apparatus 101 may be made out of materials known in the art of adhesive bandages commonly used to protect minor wounds.

The top portion of the apparatus 101 is connected to the bottom of the apparatus disposed on an opposite internal surface 102. When in the stored state, the elastic protective outer layer 101 is located on top of the opposite internal surface 102 that connects and secures removable disposable face or faces containing an abrasive surface 103, 104 on the opposite outward facing side of the face. The removable release film faces 103, 104 containing the abrasive surface on the outer facing side of the face or faces can be seen from the top view of the elastic protective outer layer 101 due to the fact that the faces 103, 104 create a line or wrinkle in the smooth elongated solid layer 101. The example shown has two disposable release film faces 103, 104 containing the abrasive surface on one side of the film face but the actual embodiment many contain any number of faces including a singular face in any variety of shapes suitable for the design and use of the adhesive bandage apparatus.

The opposite internal surface 102 comprises a layer of bandage not covered by any adhesive materials which is framed by a larger sized continuous smooth elongated solid layer with an adhesive surface 106. The bandage portion 102 of the internal surface designed to not to stick to the affected skin. The bandage portion 102 surrounded by a larger sized continuous smooth elongated solid layer 105 comprises the adhesive layer for efficiently bonding and securing the bandage to the affected skin on the internal surface of the apparatus. The adhesive layer 105 is similar to the smooth elongated solid layer 101 in that it stretches and forms to the skin surface area contour it meets. The adhesive layer 105, 106 forms a solid band around the layer of bandage 102 devoid of any adhesion in order to securely connect to the area of skin surrounding the affected skin.

The size and shape of bandage 102 typically is dependent on the size and shape of the affected skin. The larger the circumference or diameter of the affected skin, the larger the bandage 102 required to cover the skin. In the same way, the size of the adhesive layer 105 surrounding the bandage can also be larger when the surrounding skin bends or stretches itself.

For example, the shape of the apparatus 101 used on affected skin on a finger for example would require additional length on the adhesive layer 105, 106 in order to securely hold the adhesive layer 105, 106 to the skin on the finger. One skilled in the art would know the shape and size of bandages 102 used for different appendages are commonly known in the art.

Ideally, no adhesive layer 105, 106 contacts the affected area of the skin. As the adhesive layer 105, 106 meets the bandage 102 on the internal surface, a small lip forms 105 on the outer smooth protective area 101. The lip 105 is visible on the top layer 101 and gives the user a visual image of the size of the bandage 102 contained on the opposite internal surface. The lip 105 does not adversely affect any function of the bandage 102 or continuous smooth elongated solid protective layer 101.

Applicants disclose, as illustrated in FIG. 2, the bottom view or opposite view of abrasive surface filing apparatus of the present invention in a stored state. The entire outer surface 207 of the bottom of the apparatus is covered in an abrasive layer 207. The abrasive layer 207 is secured to the outer surface of the face or faces of the apparatus and covers the entire outer surface.

The entire abrasive surface 207 on the bottom of the apparatus comprised of two disposable removable faces 201, 202 from the apparatus assembly. The disposable removable faces 201, 202 comprised of an abrasive outer layer which are elongated flexible material. Each face contains an abrasive layer 201, 202 that may contain a different degrees of abrasion. In this example, section 201 contains more abrasive material to file severely affected skin areas that require more abrasive materials composed of hard components to correct and file the skin issue. While the opposite section 202 contains an abrasive material with softer components to correct and polish the skin issue. Both disposable faces 201, 202 comprise abrasive materials but the abrasive materials can vary in their function and abrasive composition.

The disposable faces 201, 202 overlap when in a resting position. The disposable faces 201, 202 cover the adhesive layer on the top of the apparatus when the bandage apparatus is not is use. The disposable faces 201, 202 meet and overlap 203, 204, 205, 206 close to the center of the bottom of the apparatus. The inner edge of the disposable faces 203, 204, 205, 206 give the user a section to easily remove the disposable faces to expose the adhesive layer. The user may peel back the abrasive layer face from either the top portion of the overlapping area 205, 206 or the lower portion of the overlapping area 203, 204.

The disposable faces 201, 202 may be moved back and forth across the affected skin or nail of the user to be filed or exfoliated. The disposable faces 201, 202 may be used while securely connected to the entire apparatus 207 or used when completely removed from the apparatus. The location of skin or nail issue may determine the desired abrasive filing face use location of the face along with the desired amount of pressure applied to the face while filing the abrasion. The abrasive face may be used connected to the bandage apparatus or removed from the apparatus. The harder the area is to reach or access, the more flexible the abrasive surface must be contorted to reach the affected area. The flexibility of the abrasive facing 201, 202 may be improved when removed from the bandage apparatus.

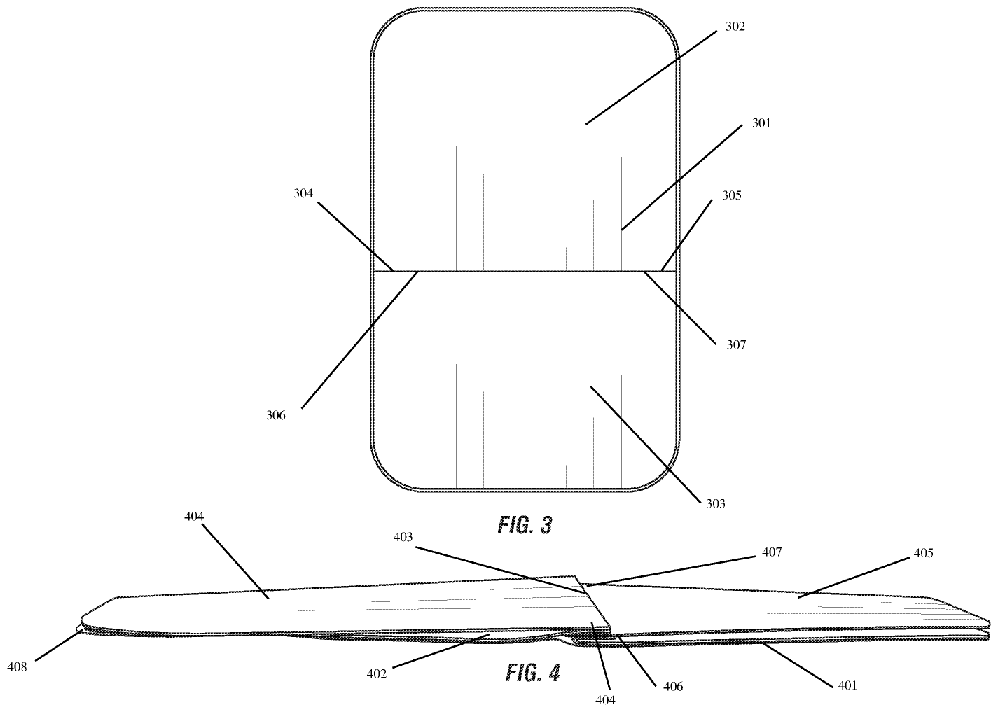

FIG. 3 is a perspective view of the abrasive surface filing apparatus of the present invention in a use position in a second embodiment. The illustration of the second embodiment of the apparatus in FIG. 3 demonstrates the bottom portion comprising the abrasive surface 301 on two faces 302, 303. This embodiment comprises two abrasive surface disposable faces comprising the same abrasive material on both faces 302, 303. FIG. 2 illustrated two faces 201, 202 with two different levels of abrasive materials for two separate filing functions. This embodiment does not have different levels of abrasion on the faces 302, 303 when the user desires the same abrasion level on each face. The present invention comprises several variations that can be altered according to user desire and skin or nail level of care. The examples outlined here are not an exhaustive list of the apparatus but are given as examples to demonstrate common uses.

Despite the same level of abrasion on the faces 302, 303 of this figure, the user employs the same mechanism to remove the abrasive face layer to expose the underneath adhesive layer as the figure comprising faces with different levels of abrasion. Separating the abrasive layer into two separate faces 302, 303 provides the user an efficient way to remove the abrasive layer 301 when the abrasive layer 301 is no longer desired or needed and to expose the bandage and adhesive contained underneath the faces. The two abrasive faces 302, 303 also protect the layer of adhesion so that the bandage and adhesive layer remain unaffected by the level of abrasion on the disposable faces 302, 303.

The second embodiment functions in the same manner as the first embodiment in that the abrasive disposable faces 302, 303 meet in the approximate middle of the adhesive bandage and over lap 304, 305, 306, 307. At the overlapping section of the apparatus 304, 305, 306, 307, the user may remove the disposable faces by peeling back on of the corners 304, 305, 306, 307 of the disposable faces 302, 303. The adhesive layer and bandage underneath the faces retains the ability to adhere to the outer surface because the disposable faces 302, 303 cover and protect the adhesive layer until desired use.

The abrasive faces illustrated on FIG. 3, are the filing apparatus that is not contained on self-adhesive bandages currently on the market. The faces on adhesive bandages currently available on the market do not contain any abrasive matter and do not have any function outside of protecting the adhesive layer. The current invention gives the bandage faces a second function and improve the overall functionality of the adhesive bandage. The bandage merges a fingernail file, pumice stone, and other smoothing manicure tools into a combination apparatus in the current invention.

The user takes the two abrasive disposable faces 302, 303 and applies the abrasive material 302, 303 to areas of the skin on user's person or a third party to smooth and shape rough areas of skin or nail. The user determines the amount of abrasion 302, 303 needed for the affected area and applies the precise amount of pressure needed to file or smooth the affected area. The user files the affected area by rubbing the abrasive material over and across the affected area until the area is smoothed to the desired softness. After the user files the affected area to the desired result, the user applies the bandage to treat with the desired pre-medicated bandage substance, which not only treats the affected skin but protects it from any outside interference. The user is able to smooth, treat and protect the skin with the current invention.

Applicants disclose, as illustrated in FIG. 4, the side view of abrasive surface filing apparatus of the present invention in a stored state. The apparatus is illustrated to show the abrasive disposable faces 404, 405 on the top section of FIG. 4. The disposable faces 404, 405 meet in the middle of the apparatus where one disposable abrasive face 403, 404 overlaps the other disposable abrasive face 406, 407.

The two disposable abrasive faces 404, 405 are shown to have two different strengths of abrasiveness on the illustration. The disposable abrasive face on the left 404 contains stronger and harder abrasive particles than the disposable abrasive face on the right 405.

Both disposable faces 404, 405 are connected to the smooth elongated solid protective layer on the opposite side 401 by a flexible solid adhesive sheet 408. The flexible sold adhesive sheet 401 comprises the solid flexible adhesive sheet 408 and a bandage 402 which is free and clear of any adhesive substance. The bandage 402 when in use protects the affected skin or area from any outside elements coming in contact with the affected area.

When the disposable abrasive faces 404, 405 are removed, the adhesive sheet 408 is exposed and ready to adhere to the skin of the user or subject requiring the apparatus. The adhesive sheet 408 secures the disposable abrasive faces 404, 405 in a fixed position so that the user can move the abrasive faces 404, 405 over the affected skin to treat, file or smooth the abrasion. Once the affected skin is treated to the users satisfaction, the disposable abrasive faces 404, 405 may be removed to reveal the adhesive layer 408.

The face resting on the top portion 403, 404 is not attached to any adhesive layer but overlaps and rests on the top portion of the other abrasive face 406, 407. This overlap 403, 404, 406, 407 provides a location for the user to easily peel back the two disposable abrasive faces one at a time. The user pulls the top layer 403, 404 of the overlap 403, 404, 406, 407 to remove the face and expose the adhesive layer. Once the faces are removed, the adhesive layer 408 may be secured by placing the bandage 402 on the desired location and pressing the adhesive layer 408 onto the user's skin.

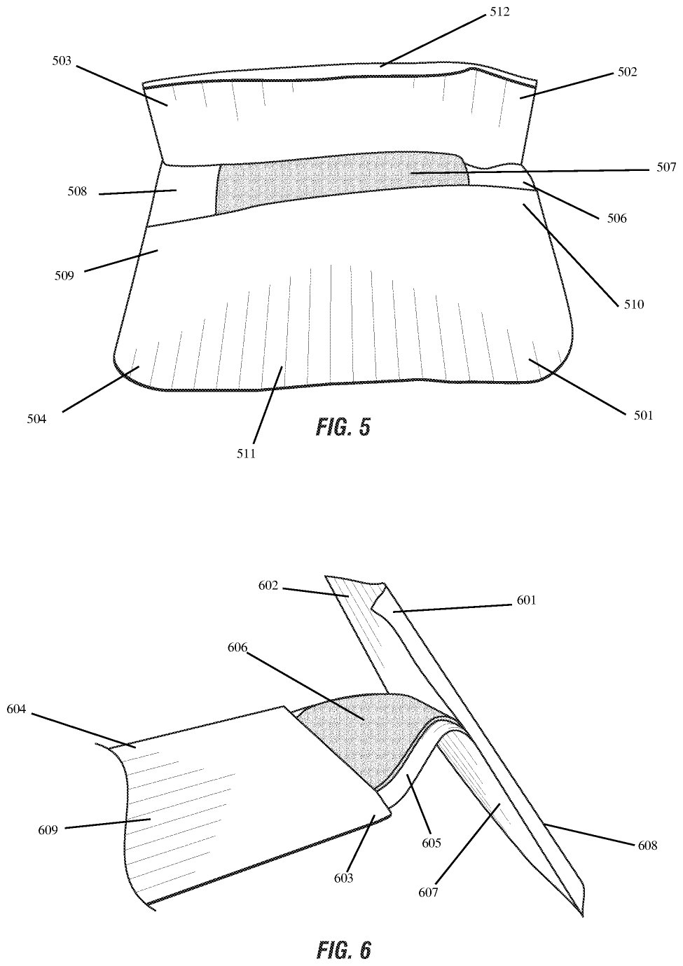

Applicants disclose, as illustrated in FIG. 5, the bottom view of abrasive surface filing apparatus of the present invention in a use position. The first disposable abrasive face 512 peels back and away from the apparatus to reveal the adhesive layer 506, 508 which held the underside of the abrasive faces in place securely on the apparatus in a stored state. The adhesive layer 506, 508 frames the perimeter of the bandage section 507 and secures the two disposable faces 511, 512 in place when in a stored state. The abrasive disposable faces 511, 512 comprised of an abrasive layer that covers the top of the elongated face, however, is not covered by the abrasive layer on the bottom layer that was in contact with the adhesive portion of the apparatus 508, 506.

The abrasive layer 502, 503 covers only the top of the elongated face in both a use and stored position. The abrasive layer 502, 503 does not contact the adhesive layer 506, 508 or bandage 507 in either the use position or stored position. The inner side of the disposable face 511, 512 is a smooth continuous polished surface that only functions to hold the faces securely in place adhering the adhesive 506, 508 when the in a stored position or when the abrasive layer is in use filing a nail or callus. The inner side of the disposable face 511, 512 does not contain any adhesive.

The abrasive face 512 as shown in the position of being removed from the apparatus, may be used as a file in the shown position. The abrasive face 512 is flexible enough for the user to bend, fold, or manipulate the face 512 into a desired flexible position to reach hard to reach areas of affected skin or nail. The user may compromise the adhesive sections 508, 506 when using the abrasive face 512 in the above described position by inadvertently allowing the abrasive particles potentially released as a result of the filing process to fall and stick to the adhesive sections 506, 508. Although the released abrasive particles may adversely affect the adhesive 506, 508, it will not destroy the adhesive 506, 508 to the point of complete failure rendering the bandage unusable.

The second disposable abrasive face 511 shown in the stored position while the first disposable abrasive face shown in the use position. After the abrasive face 512 is completely removed from the bandage apparatus, the user may then peel back the second abrasive face 511 from the apparatus by pulling on the abrasive face inner corner or corners 509, 510. As the abrasive face 511 is removed, more of the adhesive 506, 508 will be exposed in order for the remaining bandage 507 apparatus to be placed over the affected skin for protection.

Applicants disclose, as illustrated in FIG. 6, a side view of abrasive surface filing apparatus of the present invention in a use position. This figure illustrates the abrasive surface filing apparatus as the first abrasive face 608 is removed from the remaining apparatus. The user will peel back the corner of the first abrasive face 608 by pulling the inside edge 601 of the abrasive face 608.

The inside layer 602 of the removable abrasive face 608 does not contain any abrasive material. The inside layer 602 of the removable abrasive face 608 only serves to hold the abrasive face 608 in place on the bandage apparatus. The outside layer 601 of the abrasive face 608 is covered entirely in the abrasive material used to file or shape the nail or affected skin.

As the inside layer 602 and the outside layer 601 are removed, the adhesive 605 and bandage 606 are revealed and positioned for use. The top portion of the bandage apparatus 607 moves and flexes for easy removal of the abrasive face 608. The top portion of the bandage apparatus 607 as previously discussed is a cohesive, flexible, protective, pliable material that protects the affected area once placed over the affected area by user.

After the user removes the first abrasive face 608, the user then removes the second abrasive face 604 by pulling on the inside edge of the abrasive face 603. Similar to the removal of the first face, the top portion of the bandage apparatus 607 moves and flexes for easy removal of the abrasive face 604. Once both abrasive faces 608, 604 are removed, the bandage 606 and adhesive 605 are completely exposed and ready for user to apply to the affected area.

One skilled in the art would understand that the skin or nail of the user is only one example of a user of the apparatus. Because of the flexibility of the disposable faces, the apparatus could be any variety of subjects other than user. For example, the apparatus could be used on another person unable to use the apparatus themselves due to the fact the person is a child, or an adult with advancing age, or an adult with physical challenges. Additionally, the location of the affected skin could prevent the user from being able to effectively use the apparatus on themselves without third party assistance despite any physical or mental challenges.

One skilled in the art would also understand that the filing apparatus may also smooth, exfoliate, shape, scrub, level, flatten, and polish the affected skin or nail. Filing and exfoliating are used throughout the application as examples only and not meant to be an exhaustive list of filing apparatus capabilities.

Disclosed are numerous embodiments of a disposable nail file assembly with adhesive bandage that operates similar to a both a nail file and adhesive bandage when used in conjunction together but is a vast improvement in form, function, and efficacy while combining the two manicure tools into one disposable readily available inexpensive product.

It should be noted and understood that various changes and modifications to the described preferred embodiments herein will be evident to those skilled in the art. Such changes and modifications can be made without deserting from the spirit and scope of the present invention and without weakening its intended advantages. It is therefore intended that all or any such changes and modifications be enclosed by the appended claims.

Any element in a claim that does not explicitly state “means for” performing a specified function, or “step for” performing a specific function, is not to be interpreted as a “means” or “step” clause as specified in 35 U.S.C. § 112, ¶ 6. In particular, the use of “step of” in the claims herein is not intended to invoke the provisions of 35 U.S.C. § 112, 16.

The invention claimed is:

1. A file assembly comprising:
A flexible face having a first surface and an opposite second surface;
an abrasive layer secured to the first surface of a flexible face; and
an adhesive bandage apparatus connected at least in part by the second surface.

2. The file assembly set forth in claim 1 where in the abrasive layer covers the entire surface area of the first surface.

3. The file assembly set forth in claim 1 where the adhesive bandage comprises a gauze like material and adhesive layer.

4. The file assembly set forth in claim 1 where in the entire assembly is disposable in whole and in part.

5. The file assembly set forth in claim 1 where in the second surface of the flexible face is a smooth surface devoid of any abrasive material.

6. The file assembly set forth in claim 1 where in the second surface connects the flexible face to the bandage apparatus by a layer of adhesive contained on the bandage apparatus.

7. The file assembly set forth in claim 1 where in the second surface further comprises a release liner for the adhesive bandage.

8. The file assembly set forth in claim 1 where in the flexible face is divided into two separate faces.

9. The file assembly set forth in claim 1 where in the flexible face is divided into more than two separate faces.

10. The file assembly set forth in claim 1 where in the bandage apparatus comprises a bandage that is pretreated with a sanitization agent.

11. The file assembly set forth in claim 1 where the bandage apparatus comprises a bandage that is pretreated with a moisturizing agent.

12. The file assembly set forth in claim 1 where in the bandage apparatus comprises a bandage that is pretreated with a first aid ointment.

13. The file assembly set forth in claim 1 where in the abrasive layer comprises granular abrasive particulates.

14. The file assembly set forth in claim 1 where the abrasive layer comprises granular abrasive particulates ranging in size from 40 to 1200 grit.

15. A method of utilizing a file assembly comprising the steps of:
securing flexible face comprising an outward facing abrasive layer connected to an adhesive bandage;
moving the outward facing abrasive layer connected to the adhesive bandage directly over and across to file and smooth the affected area of skin or nail;
removing the flexible face from the adhesive bandage after desired filing and smoothing;
removing the flexible face from the apparatus to reveal adhesive layer of bandage apparatus;
and applying bandage portion of apparatus to directly to affected area of skin by placing the adhesive section of the bandage apparatus on unaffected skin surrounding the affected skin.

16. The method of claim 15 where the abrasive layer comprises granular abrasive particulates.

17. The method of claim 15 where the abrasive layer comprises granular abrasive particulates ranging in size from 40 to 1200 grit.

18. The method of claim 15 where in the flexible face is divided into two separate faces.

19. The method of claim 15 where in the flexible face is divided into more than two separate faces.

20. The method of claim 15 where the bandage apparatus comprises a bandage that is pretreated with a skin treatment agent.
